(19) United States
(12) Patent Application Publication
(10) Pub. No.: US 2005/0256092 A1
(43) Pub. Date: Nov. 17, 2005

(54) Antipsoriatic agent

(76) Inventor: Shin Shimaoka, SHIZUOKA (JP)

Correspondence Address: BROWDY AND NEIMARK, P.L.L.C.;624 NINTH STREET, NW, SUITE 300, WASHINGTON, DC, 20001-5303, US

(21) Appl. No.: 10/522,886

(22) PCT Filed: Aug. 1, 2003
(86) PCT No.: PCT/JP03/09814

§ 371 Date: Feb. 1, 2005

(30) Foreign Application Priority Data
Aug. 1, 2002 (JP) 2002-224297

(51) Int. Cl. A61K 31/59; C07C 401/00

(52) U.S. Cl. 514/167; 552/653

(57) ABSTRACT

An object of the present invention is to synthesize a pharmaceutical effective for the treatment of psoriasis.

A therapeutic agent for psoriasis, comprising 2β-(3-hydroxypropyloxy)-1α,25-dihydroxyvitamin D₃ as an active ingredient, is provided by the present invention.

### TECHNICAL FIELD

This invention relates to a therapeutic agent for psoriasis, comprising a vitamin D derivative as an active ingredient.

### BACKGROUND ART

Psoriasis is a chronic intractable skin disease, characterized by abnormal proliferation of skin cells. Its etiology is not yet clear, but the deviation of skin cells from the normal growth mechanism and differentiation mechanism is considered to be a main cause. There has been an increase in the number of cases of psoriasis in recent years, and most psoriatic cases involve well-demarcated papules or erythemas with thick scales, and follow a chronic course. This type of psoriasis is called psoriasis vulgaris. Unlike psoriasis vulgaris, psoriasis pustulosa forms pustules on erythemas. Psoriasis pustulosa is classified into generalized (Zumbush's) pustular psoriasis which occurs over wide areas and involves systemic symptoms, and localized (Barber's) pustular psoriasis which develops over small areas, such as the hands or feet. Psoriasis may occasionally cause redness, swelling, degeneration or ankylosis of joints of the hands or feet, elbow and knee. This is called arthritic psoriasis.

Treatments for psoriasis include external application of corticosteroids, photochemotherapy (PUVA), and oral administration of retinoids. However, these treatments have not always had a satisfactory therapeutic effect. In recent years, 1α,25-dihydroxyvitamin D₃, calcipotriol, etc., which are known as active vitamin D₃, have been shown to have the activity of suppressing the proliferation of keratinocytes, and to be useful as therapeutic agents for psoriasis (European Patent Publication No. 129003, “Vitamin D in Dermatology”, edited by Knud Kragballe (2000), Marcel Dekker Inc., and Drugs 43(3), 415-429 (1992)). However, more potent and more effective pharmaceuticals are still desired.

2β-(3-Hydroxypropyloxy)-1α,25-dihydroxyvitamin D₃, which is a vitamin D derivative having a substituent at the 2-position, is known to have a calcium regulating action (JP 61-267549 A) and an osseous union promoting action (JP 08-12580 A). However, its use as a therapeutic agent for psoriasis has not been known at all.

### DISCLOSURE OF THE INVENTION

As described above, existing treatment methods and therapeutic agents for psoriasis have not been entirely satisfactory, and more potent and effective treatments and therapeutic drugs have been desired. It is an object of the present invention to provide an effective therapeutic agent and treating method for psoriasis.

The inventor of the present invention investigated the suppressing action of 2β-(3-hydroxypropyloxy)-1α,25-dihydroxyvitamin D₃ on the keratinocyte proliferation. The inventor has found that surprisingly this compound has a very potent suppressing action, in comparison with active vitamin D₃, and has accomplished the present invention.

That is, the present invention provides a therapeutic agent for psoriasis, which agent comprises a compound represented by the following Formula (I) 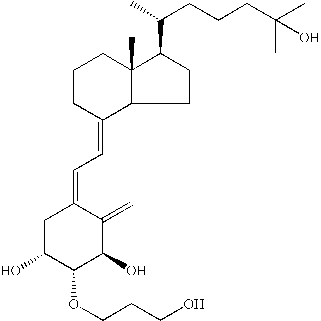
as an active ingredient.

The therapeutic agent for psoriasis according to the present invention preferably suppresses the proliferation of keratinocytes.

The therapeutic agent for psoriasis according to the present invention may be administered to animals as well as humans.

According to another aspect of the present invention, there is provided a method for the treatment of psoriasis in a human or an animal, the method comprises administering a therapeutically effective amount of a compound represented by the following Formula (I) 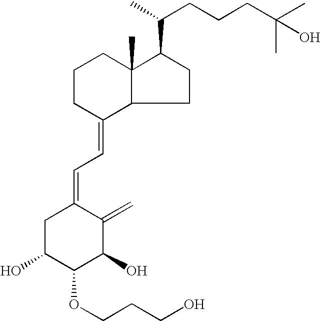
to a human or an animal in need of such treatment.

According to still another aspect of the present invention, there is provided use of a compound represented by the following Formula (I) 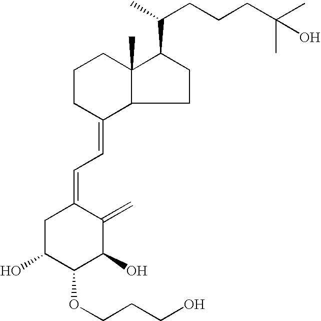
in production of a therapeutic agent for psoriasis.

### BRIEF DESCRIPTION OF THE DRAWING

FIG. 1 is a graph showing the effect of suppressing the proliferation of cultured human keratinocytes by an active vitamin D₃ (designated as “1,25D3” in the figure) and 2β-(3-hydroxypropyloxy)-1α,25-dihydroxyvitamin D₃ (designated as “ED-71” in the figure). In the figure, filled rhombuses (♦) represent the active vitamin D₃, and open circles (◯) represent ED-71.

### PREFERRED MODE FOR CARRYING OUT THE INVENTION

The entire disclosure of Japanese Patent Application No. 2002-224297, an application as the basis for priority claimed by the present application, is incorporated herein by reference in its entirety.

The compound represented by the Formula (I), namely, 2β-(3-hydroxypropyloxy)-1α,25-dihydroxyvitamin D₃, can be synthesized, for example, by the method described in JP 61-267549 A, although the method for its synthesis is not limited.

The therapeutic agent for psoriasis according to the present invention can be administered orally, parenterally (subcutaneous injection, intravenous injection, intramuscular injection, intraperitoneal injection, etc.), enterally, or topically. Topical administration, such as by an agent for external use, is preferred, but systemic administration as an oral agent or an injection may be performed. It is also possible to use a mode of administration, such as oral administration, injection, or external use, in a suitable combination.

The therapeutic agent for psoriasis according to the present invention may contain a pharmaceutically acceptable carrier or diluent in addition to the active ingredient. Examples of the carrier or diluent are vehicles (starch, lactose, etc.), disintegrants (alginic acid, etc.), tablet lubricants (stearic acid, talc, etc.), binders (starch, etc.), antioxidants (ascorbic acid, etc.), emulsifiers (polysorbate, etc.), surfactants (sorbitan monoesters, etc.), preservatives (benzoic acid), perfumes, and colorants. Other therapeutic ingredients may be contained further.

The therapeutic agent for psoriasis according to the present invention can be appropriately formulated according to the route of administration, such as oral administration, enteral administration, parenteral (including subcutaneous, intramuscular, and intravenous) administration, or external use.

For oral administration, such formulations as tablets, capsules, powders, granules, syrups, and elixirs are available. For parenteral administration, such formulations as injections (e.g., liquids or suspensions) are available. For external use as topical administration, such formulations as ointments, creams and lotions are available. For enteral administration, such formulations as suppositories and enemas are available.

The dose of the therapeutic agent for psoriasis in the present invention can be selected, as appropriate, according to the state of disease, the body weight and age of a subject to be treated, the route of administration and the dosage form of the agent of the present invention. For administration to animals, the dose is greatly affected by the body weight of individual animals. In the human adult, the usual oral dose of 2β-(3-hydroxypropyloxy)-1α,25-dihydroxyvitamin D₃, as the active ingredient, can be selected from the range of 0.0001 μg to 1,000 μg, preferably 0.001 μg to 100 μg, more preferably 0.01 μg to 10 μg, most preferably 0.1 μg to 1 μg, per day, and this dose can be used once daily or as two to three divided doses per day. For external medicine, such as an ointment, the dose of this compound as the active ingredient can be selected from the range of 0.0001 μg to 10,000 μg, preferably 0.001 μg to 1,000 μg, more preferably 0.01 μg to 100 μg, most preferably 0.1 μg to 25 μg, per day.

### EXAMPLES

The present invention will be described in further detail by the following Examples and Manufacturing Examples.

### Example 1

The effect of suppressing the proliferation of cultured human keratinocytes by 2β-(3-hydroxypropyloxy)-1α,25-dihydroxyvitamin D₃ (hereinafter referred to as “ED-71”) was investigated.

KGM-2 culture medium was added to each well of a 96-well plate (COSTAR 3595), and adult-human-derived keratinocytes (Clonetics) were seeded at a cell count of 1×10³/well. Then, active vitamin D₃ (1α,25-dihydroxyvitamin D₃, produced by Solvay Pharmaceuticals) or ED-71 (produced by Chugai Seiyaku) was added to each well in a final concentration of 1×10⁻¹⁰ mol/L, 1×10⁻⁹ mol/L, 1×10⁻⁸ mol/L, or 1×10⁻⁷ mol/L. The cells were cultured in the KGM-2 culture medium at a cell concentration of 1×10³/200 μl/well for 3 days at 37° C. in an atmosphere of 5% CO₂ and 95% air. [³H]thymidine was added in an amount of 7.4 kBq/well, and the cells were further cultured for 1 day. The culture medium was removed, and the cells were stripped off using 0.05% trypsin/EDTA (GIBCO BRL), and the amount of [³H]thymidine taken up by the cells was measured with a liquid scintillation counter (1450 MICROBETA, Wallac). The cells cultured and treated in the same manner as described above, except for the addition of the active vitamin D₃ or ED-71, were used as a control.

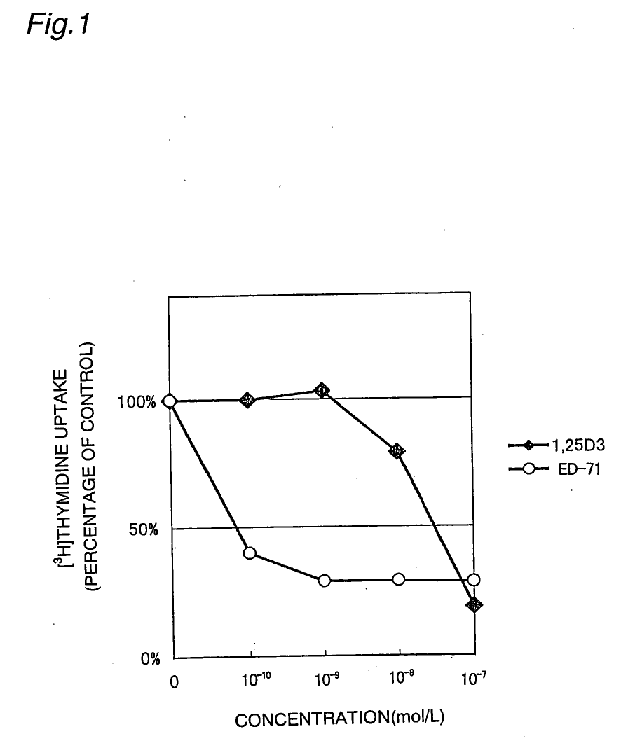

The results are shown in FIG. 1. In FIG. 1, the [³H]thymidine uptake into the cells treated with each drug is expressed as a percentage of the [³H]thymidine uptake into the control cells.

As shown in FIG. 1, the IC₅₀ (mol/L) value of the active vitamin D₃ was 3.05×10⁻⁸ mol/L, while the IC₅₀ (mol/L) value of ED-71 was <1.0×10⁻¹⁰ mol/L.

In accordance with the following calculation equation, the human keratinocyte proliferation suppressing activity of ED-71 was calculated as a relative value with respect to the active vitamin D₃. This activity was found to be 305.23 or more.
Relative value=(IC₅₀ value of active vitamin D₃)/(IC₅₀ value of ED-71)
This outcome shows that ED-71 has a very potent keratinocyte proliferation suppressing action, as compared with active vitamin D₃.

### Example 2

The effect of ED-71 administered percutaneously and orally was investigated using hairless mice.

Percutaneous administration of a vitamin D₃ derivative in hairless mice was reported to cause hyperplasia of the epidermis (British Journal of Dermatology 1995; 132; 841-852). Following a single percutaneous dose of active vitamin D₃ (1α,25-dihydroxyvitamin D₃) and ED-71 administered to hairless mice, ED-71 thickened the epidermis in a lower dose than the dose of active vitamin D₃. When active vitamin D₃ and ED-71 were administered orally to hairless mice for 4 days, ED-71 thickened the epidermis in a lower dose than the dose of active vitamin D₃. These results suggested that ED-71, administered percutaneously or orally, would be effective.

### Preparation Example 1

ED-71 (0.5 mg) is mixed with a hydrophilic ointment having the following formulation to obtain a hydrophilic ointment containing 0.5 μg of ED-71 per gram:

| White petrolatum | 250 | g |
|---|---|---|
| Stearyl alcohol | 220 | g |
| Propylene glycol | 120 | g |
| Sodium lauryl sulfate | 15 | g |
| Ethyl parahydroxybenzoate | 0.25 | g |
| Propyl parahydroxybenzoate | 0.15 | g |
| Purified water | appropriate amount | |
| Total amount | 1000 | g |

### Preparation Example 2

ED-71 (1.0 mg) is dissolved in 60 g of a triglyceride of a middle chain fatty acid, and 30 mg of sorbic acid is added as a stabilizer. The mixture is processed in accordance with a conventional method using a gelatin shell soft capsule manufacturing machine to obtain soft capsules containing 1.0 μg of ED-71 per capsule.

### INDUSTRIAL APPLICABILITY

As described above, 2β-(3-hydroxypropyloxy)-1α,25-dihydroxyvitamin D₃ has an excellent keratinocyte proliferation suppressing action. The therapeutic agent of the present invention, comprising this compound as an active ingredient, is expected to be useful for treatment of psoriasis.

1. A therapeutic agent for psoriasis, comprising a compound represented by the following Formula (I) 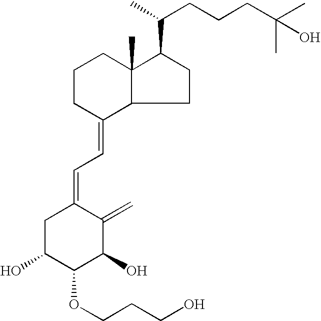
as an active ingredient:

2. The therapeutic agent for psoriasis according to claim 1, said therapeutic agent suppressing proliferation of keratinocytes.

3. A method for the treatment of psoriasis, comprising administering an effective amount of a compound represented by the following Formula (I) 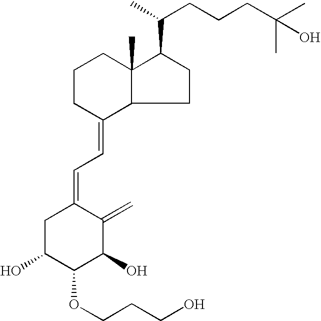
to a subject in need of such treatment.

4. The method according to claim 3, wherein proliferation of keratinocytes is suppressed.
